(19) United States
(12) Patent Application Publication
(10) Pub. No.: US 2012/0191144 A1
(43) Pub. Date: Jul. 26, 2012

(54) INSTRUMENT FOR REDUCTION OF A VERTEBRAL ROD AND METHOD OF USE

(75) Inventors: Bertrand Peultier, Les Hopitaux Neufs (FR); Loic Josse, Yens (CH); Jason M. May, Cordova, TN (US)

(73) Assignee: WARSAW ORTHOPEDIC, INC, Warsaw, IN (US)

(21) Appl. No.: 13/014,129

(22) Filed: Jan. 26, 2011

(51) Int. Cl. A61B 17/70; A61B 17/88

(52) U.S. Cl. 606/86 A; 606/279

(57) ABSTRACT

An instrument and method for reducing a vertebral rod into an anchor attached to a vertebral member. The instrument may include a gripper configured to attach to an anchor, an elongated shaft extending along a longitudinal axis between opposing jaws of the gripper, and a reducing element disposed at least partially outside the first and second jaws of the gripper and configured to engage with the vertebral rod. A release device may also be disposed about the elongated shaft and may be movable relative thereto such that the release device may be selectively engaged with a threaded outer surface of the elongated shaft. The reducing element may therefore be alternatively slidable relative to the gripper or threadably movable relative to the gripper by interaction with the elongated shaft, depending on a position of the release device.

### BACKGROUND

The present application is directed to instruments and methods for moving a vertebral rod within a patient and into a bone anchor attached to a vertebral member and, more particularly, to instruments with a release device to facilitate rapid movement of a reducing element toward the vertebral rod followed by steady sequential movement of the vertebral rod into the bone anchor.

The spine is divided into four regions comprising the cervical, thoracic, lumbar, and sacrococcygeal regions. The cervical region includes the top seven vertebral members identified as C1-C7. The thoracic region includes the next twelve vertebral members identified as T1-T12. The lumbar region includes five vertebral members L1-L5. The sacrococcygeal region includes nine fused vertebral members that form the sacrum and the coccyx. The vertebral members of the spine are aligned in a curved configuration that includes a cervical curve, thoracic curve, and lumbosacral curve. Intervertebral discs are positioned between the vertebral members and permit flexion, extension, lateral bending, and rotation.

Elongated members may be attached to the vertebral members to various reasons such as to provide support, redirect stresses over a wider area away from a damaged or defective region, and restore the spine to its proper alignment. The elongated members are secured to one or more vertebral members through connectors. The connectors include a receiver that receives the elongated member, and an anchor to anchor into the vertebral member.

Instruments are needed to insert or reduce the elongated members into the anchors. The instruments should be sized and shape for insertion into the patient and connection to the bone anchors. The instruments should be designed for effective and efficient movement of the vertebral rod into the anchor.

### SUMMARY

The present application discloses instruments and methods for moving a vertebral rod into a bone anchor that is attached to a vertebral member. The instrument may include a gripper with opposing first and second jaws. The jaws may include distal ends that may be configured to attach to the bone anchor. The instrument may also include an elongated shaft extending along a longitudinal axis, wherein the elongated shaft is operably engaged with the gripper. The elongated shaft may include a threaded outer surface and the elongated shaft. The instrument may also include a reducing element disposed at least partially about an outer surface of the first and second jaws of the gripper and configured to engage with the vertebral rod. The instrument may also include a release device disposed about the elongated shaft, wherein the release device is movable between a first position and a second position relative to the elongated shaft. When the release device is in the first position, the release device may be substantially disengaged from the threaded outer surface of the elongated shaft such that the reducing element is slidable along the longitudinal axis relative to the gripper. Furthermore, when the release device is in the second position, the release device may be threadably engaged with the threaded outer surface of the elongated shaft such that a relative movement of the elongated shaft and the reducing element along the longitudinal axis may move the vertebral rod towards the distal ends of the gripper and into the anchor.

The various aspects of the various embodiments may be used alone or in any combination, as is desired.

### BRIEF DESCRIPTION OF THE DRAWINGS

FIG. 1 is a front view of a reduction instrument according to one embodiment, shown relative to an anchor and vertebral rod.

FIG. 2 is a side view of a reduction instrument according to one embodiment, shown relative to an anchor and vertebral rod.

FIG. 3 is a section view of the reduction instrument shown in FIG. 2, shown relative to an anchor and vertebral rod.

FIG. 4 is a detailed perspective view of the proximal end of the reduction instrument shown in FIGS. 1-3.

FIG. 5 is a detailed section view of the proximal end of the reduction instrument shown in FIGS. 1-4.

FIG. 6 is a perspective view of a reduction instrument according to one embodiment.

FIG. 7 is a front view of a reduction instrument according to one embodiment.

FIG. 8 is a section view of the reduction instrument shown in FIG. 7.

FIG. 9 is a partial front section view of the reduction instrument shown in FIGS. 6-8, shown relative to an anchor, vertebral rod and set fastener, including a set fastener driver extending through a bore defined in the reduction instrument.

FIG. 10 is a front section view of the reduction instrument shown in FIGS. 6-8, shown relative to an anchor, including a driver extending through a bore defined in the reduction instrument for driving the anchor into a vertebral body.

FIG. 11A is a perspective view of the gripper and release device components of a reduction instrument according to one embodiment.

FIG. 11B is an exploded perspective view of the gripper and release device components shown in FIG. 11A.

FIG. 12A is a perspective view of the elongated shaft and reducing element components of a reduction instrument according to one embodiment.

FIG. 12B is an exploded perspective view of the elongated shaft and reducing element components shown in FIG. 12A.

FIG. 13 is an exploded perspective view of the subcomponent assemblies shown in FIGS. 11A and 12A, according to one embodiment.

FIG. 14A is a detailed front view of the distal end of a reduction instrument, according to one embodiment.

FIG. 14B is a front section view of the distal end of the reduction instrument shown in FIG. 14A.

FIG. 15A is a perspective view of a reduction instrument according to one embodiment, wherein the reducing element is disposed at a substantially proximal position relative to the gripper.

FIG. 15B is a perspective view of a reduction instrument according to one embodiment, wherein the reducing element is disposed at a substantially medial position relative to the gripper.

FIG. 15C is a perspective view of a reduction instrument according to one embodiment, wherein the reducing element is disposed at a substantially distal position relative to the gripper.

FIG. 16 is a perspective view of a reduction instrument shown relative to an anchor and vertebral rod, according to one embodiment.

FIG. 17 is a perspective section view of the reduction instrument shown in FIG. 16, shown relative to an anchor and vertebral rod (also in section);

FIG. 18 is a front view of a reduction instrument according to one embodiment;

FIG. 19 is a front section view of the reduction instrument shown in FIG. 18;

FIG. 20 is a perspective view of a reduction instrument shown relative to an anchor and vertebral rod, according to one embodiment;

FIG. 21 is a perspective view of the detail XXI highlighted in FIG. 20, showing the release device, according to one embodiment;

FIG. 22 is a perspective view of certain components of the reduction instrument shown in FIG. 20;

FIG. 23 is a perspective view of a reduction instrument shown relative to an anchor and set fastener, according to one embodiment;

FIG. 24 is a perspective view of a reduction instrument shown relative to an anchor, according to one embodiment;

FIG. 25 is a perspective view of a reduction instrument shown relative to an anchor and set fastener, according to one embodiment;

FIG. 26 is a perspective view of a reduction instrument shown relative to an anchor and set fastener, according to one embodiment; and

FIG. 27 is a perspective view of a reduction instrument shown relative to an anchor and set fastener, according to one embodiment.

### DETAILED DESCRIPTION

The present application is directed to instruments and methods for reduction of a vertebral rod into an anchor attached to vertebral members. One type of anchor is the CD-Horizon Legacy Fixed Angle Screw manufactured by Medtronic Sofamor Danek of Memphis, Tenn. Other anchors may also be used, including but not limited to: fixed angle screws, multi-axial screws, iliac screws, and a variety of other spinal instrumentation (including revision connectors).

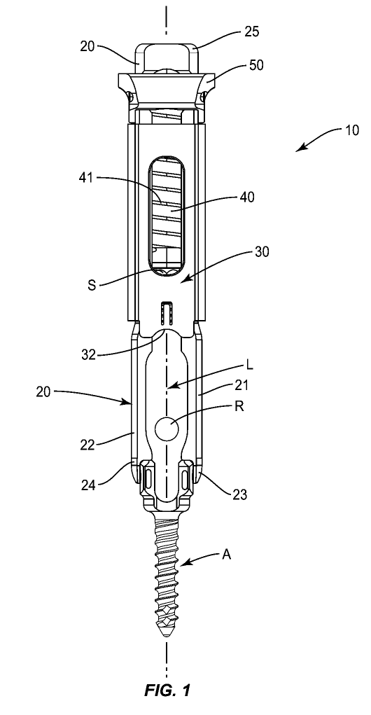

Referring to FIG. 1, the terms “proximal” and “proximally” are used herein to generally denote a position away from the patient and towards a clinician operating the instrument 10 and/or a direction along the axis L away from the distal ends 21, 23 of the instrument 10 and/or opposite the end of the instrument 10 that is attached to the anchor A. Furthermore, the terms “distal” and “distally” are used herein to generally denote a position away from the clinician operating the instrument 10 and towards a patient and/or a direction along the axis L towards the distal ends 21, 23 of the instrument 10 and/or towards the end of the instrument 10 that is attached to the anchor A.

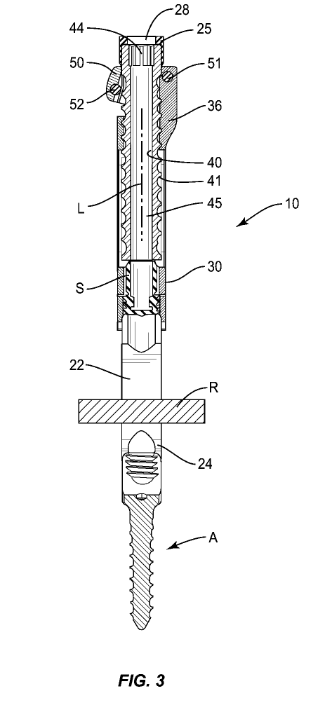

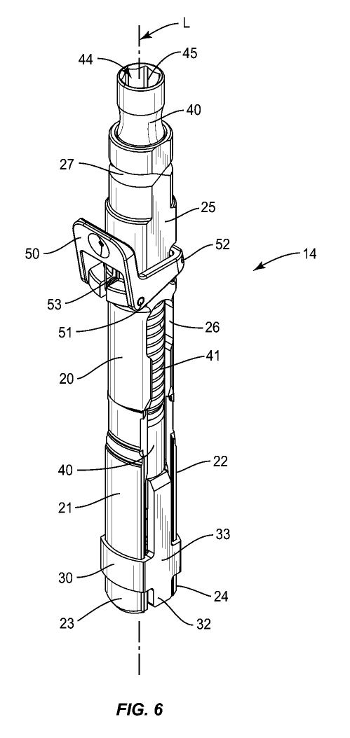

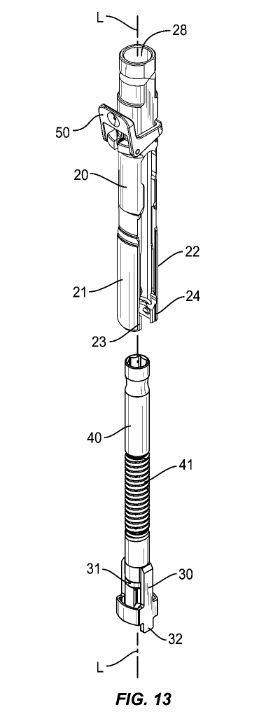

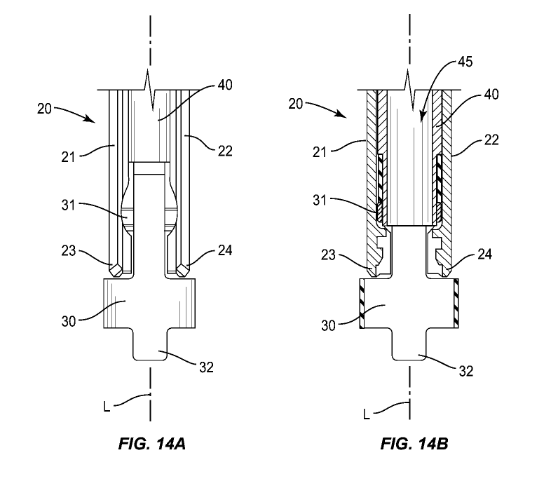

FIG. 1 includes an embodiment of an instrument 10 for moving a vertebral rod R into an anchor A attached to a vertebral member (not shown). The instrument 10 may comprise a gripper 20 including opposing first and second jaws 21, 22 each with a distal end 23, 24, respectively, to attach to the anchor A. The instrument 10 may also comprise a proximal end and/or a transverse member 25, shown in FIG. 1. The transverse member 25 may connect the first and second jaws 21, 22. Furthermore, in some embodiments described herein (such as the instrument 14 shown in FIGS. 6-15C) the transverse member 25 may define an aperture 26 through which the elongated shaft 40 extends. In other embodiments, as shown generally in FIG. 3, the transverse member 25 may also define an aperture 28 sized to allow a drive element (such as a hex driver) to pass therethrough to engage a drive interface 44 defined at a proximal end of the bore 45 defined in the elongated shaft 40.

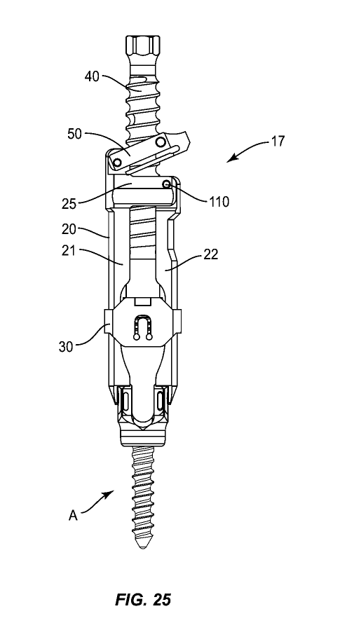

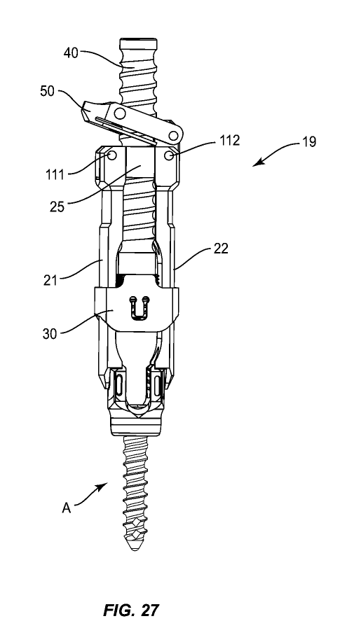

In some embodiments, the gripper 20 may comprise first and second jaws 21, 22 that are hinged relative to transverse member 25. For example, FIG. 25 shows an embodiment of instrument 17 wherein the first jaw 21 is formed as an integral extension of the transverse member 25 of the gripper 20, while the second jaw 22 is pivotable about a hinge 110 connecting the second jaw 22 with the transverse member 25. The gripper 20 may also be formed as a single piece that does not include separate jaws 21, 22. FIG. 27, shows an embodiment of instrument 19 wherein the first and second jaws 21, 22 are each pivotable about hinges 111, 112, respectively. The hinges 111, 112 connect each of the first and second jaws 21, 22 with the transverse member 25.

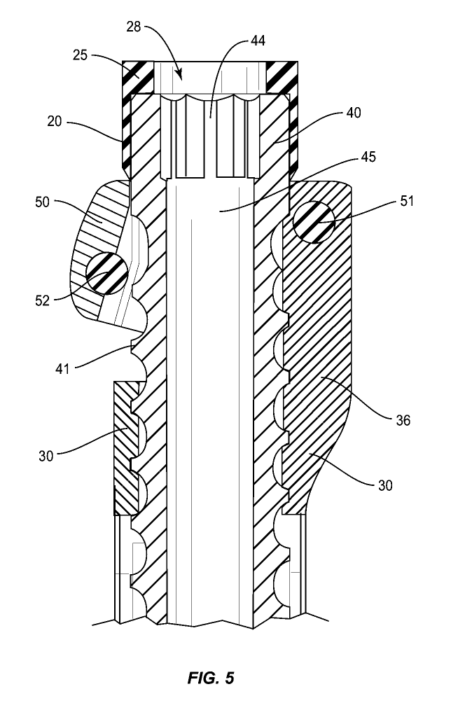

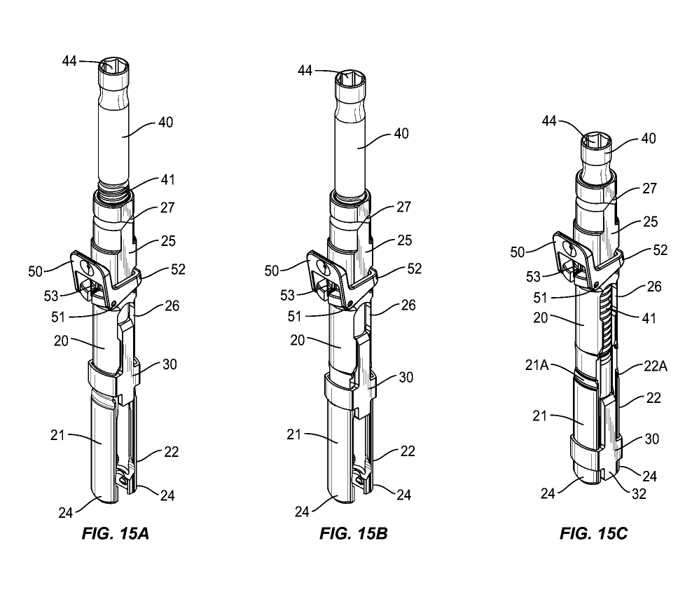

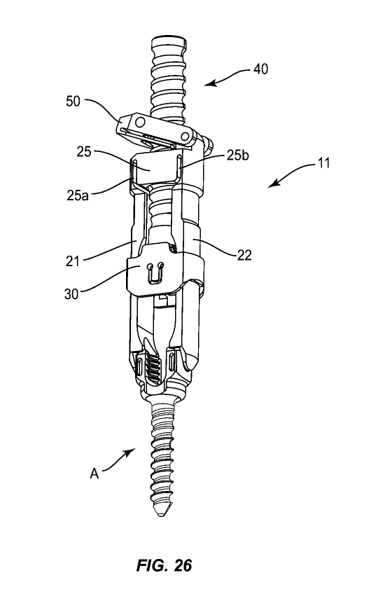

In other embodiments, the gripper 20 may comprise jaws 21, 22 formed as a single, unitary body with the transverse member 25 and/or other components of the gripper 20. For example, FIG. 26 shows an embodiment of instrument 11 wherein both the first and second jaws 21, 22 are formed as integral extensions of the transverse member 25 of the gripper 20. To provide for some flexibility, instrument 11 may comprise a transverse member 25 defining a pair of flex slots 25a, 25b such that the integrally formed first and second jaws 21, 22 may be urged slightly toward and/or away from one another by a spring bias or by the action of the reducing element 30 (which, as described herein, may be disposed at least partially about an outer surface of the first and second jaws 21, 22 of the gripper 20) as it is moved distally towards the distal ends 23, 24 of the jaws 21, 22. An example of the progression of the reducing element 30 distally along the longitudinal axis L is shown in the series of FIGS. 15A-15C. FIGS. 1-15C also show various embodiments of instruments 10, 14 wherein the jaws 21, 22 are formed as substantially integral extensions of the transverse member 25 of the gripper 20.

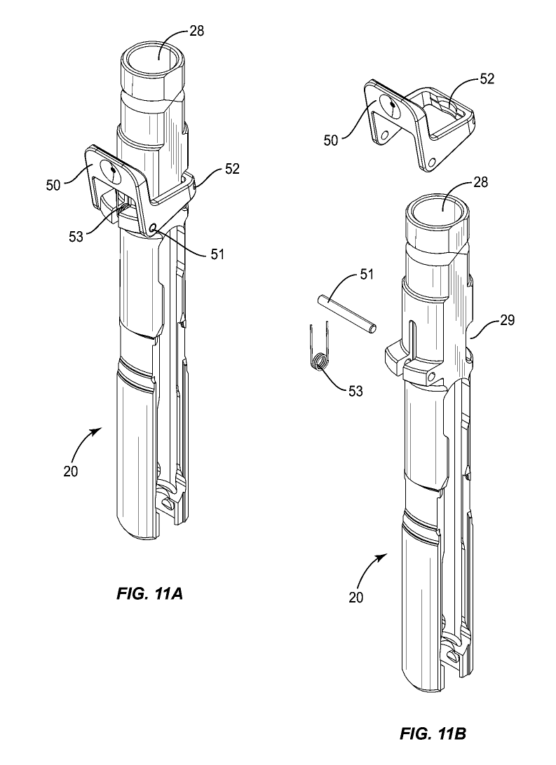

Referring generally to FIGS. 15A-15C, the jaws 21, 22 of the gripper 20 may, in some embodiments, be formed and/or configured to be biased away from one another as they extend distally from the transverse member 25 (see, for example, the gripper 20 component shown in exploded view in FIG. 11B). As described herein, the reducing element 30 is disposed at least partially about an outer surface of the first and second jaws 21, 22 of the gripper 20. A cutout 26 may be defined in the gripper 20 body to define a level of flexibility in the gripper. A variety of specific geometries of the cutout may be selected to modify the flexibility and/or bias of the various components of the gripper 20. For example, when the reducing element 30 is positioned proximally along the gripper, the jaws 21, 22 may be formed so as to flex naturally away from one another (see FIGS. 15A) such that an anchor A may be placed between the distal ends 23, 24 of the jaws 21, 22.

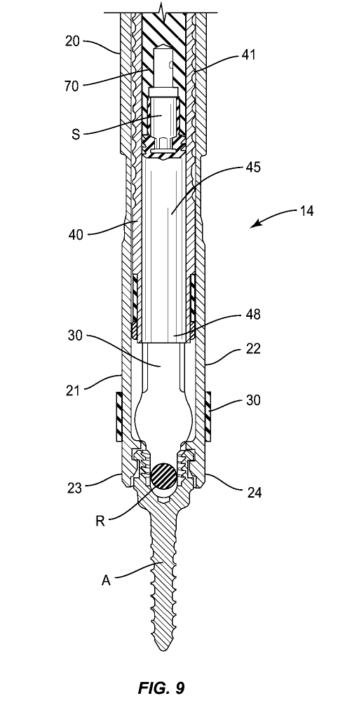

Referring now to FIG. 15B, as the reducing element 30 is moved distally over the shoulders 21A, 22A formed on the jaws 21, 22, the reducing element 30 (being disposed at least partially about an outer surface of the first and second jaws 21, 22) urges the jaws 21, 22 toward one another so that they may grip an anchor A therebetween (see, for example, FIG. 9). In some embodiments, the distal ends 23, 24 of the jaws 21, 22 may be provided with tabs or other extensions configured to fit into complementary recesses formed in a proximal portion of anchor A (such as the “tulip” of a fixed or multiaxial pedicle screw) in order to more firmly grip the anchor A when the gripper 20 is in the “closed” position shown generally in FIG. 15B.

As described more fully herein, the reducing element 30 shown in FIGS. 15A-15C may be moved by sliding the elongated shaft 40 relative to the gripper 20 when the release device 50 is disengaged from the threads 41 formed on the outer surface of the elongated shaft 40. This sliding action may be used, for example, to move the gripper 20 from the open position (see FIG. 15A) to the closed (or “anchor-gripping”) position shown in FIG. 15B. As described herein, the reducing element may also be moved distally (and/or proximally) relative to the gripper 20 by rotating the elongated shaft 40 (using the drive interface 44, for example) when the release device 50 is engaged with the threads 41 formed on an outer surface of the elongated shaft 40. This rotational (or screw action) drive may be used to more slowly and steadily drive the reducing member 30 towards the distal ends 23, 24 of the gripper 20 in order to reduce a rod R into an anchor A that may be gripped between the jaws 21, 22 of the instrument. This “fully reduced” position (wherein the reducing element 30 is moved distally such that a reducing tab 32 is substantially even with the distal ends 23, 24) is shown in FIG. 15C. The instrument 10 may also further comprise an elongated shaft 40 disposed at least partially within the gripper 20 and extending along a longitudinal axis L between the opposing first and second jaws 21, 22. In some embodiments, the elongated shaft 40 includes a threaded outer surface comprising a plurality of threads 41. In some embodiments, the elongated shaft 40 may be substantially fixed relative to the gripper 20 along the longitudinal axis L (see, for example, FIGS. 1-5 and 16-17).

Furthermore, the elongated shaft 40 may be rotatable relative to the gripper 20 about the longitudinal axis L. For example, the elongated shaft 40 may be housed in a proximal portion of the gripper 20 as shown generally in FIG. 1 and seated in a circular housing formed in the transverse member 25 such that the elongated shaft 40 may be rotatable within a proximal portion of the gripper 20 about the longitudinal axis L. In such embodiments wherein the elongated shaft is rotatable relative to the gripper 20 about the longitudinal axis but not slidable relative to the gripper 20 along the axis L, the reducing element 30 may be slidable about an outer surface of the gripper 20 and selectively engageable with the threads 41 of the elongated shaft 40 via the release device 50.

In other embodiments, the elongated shaft 40 may be substantially non-rotatable relative to the gripper 20 about the longitudinal axis L. For example, the elongated shaft 40 may be housed in a proximal portion of the gripper 20 as shown generally in FIG. 1 and non-rotatably seated in a housing formed in the transverse member 25 such that the elongated shaft 40 may be substantially fixed within a proximal portion of the gripper 20. In some such embodiments, the reducing element 30 may be directly attached to the release device 50, which may be rotated about the fixed elongated shaft 40 in order to drive the reducing element 30 towards the distal ends 23, 24 of the gripper jaws 21, 22 when the release element 50 is in the second position as described further herein.

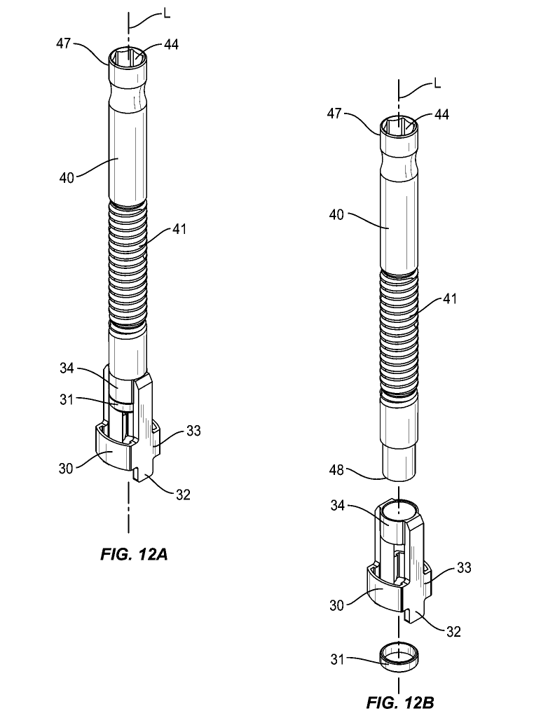

In other embodiments, such as those shown generally in FIGS. 6-8 and 18-27, the elongated shaft 40 may be movable relative to the gripper 20 along the longitudinal axis L. For example, the elongated shaft 40 may be slidable relative to the gripper 20 along the longitudinal axis L and extend proximally outward from the gripper 20 through an aperture 28 defined in the transverse member 25 of the gripper 20. In such embodiments wherein the elongated shaft 40 is selectively slidable relative to the gripper 20, the reducing element 30 may be attached directly to a distal end 48 of the elongated shaft 40 (as shown, for example, in FIGS. 12A and 12B).

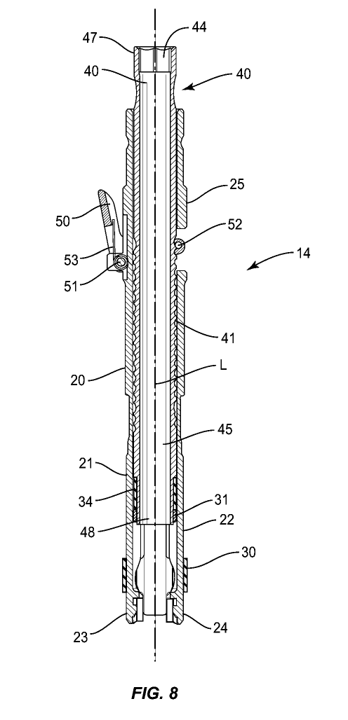

In some embodiments as shown, for example in FIGS. 3 and 8, the elongated shaft 40 may define a bore 45 extending therethrough along the longitudinal axis L. As shown in FIG. 3, the bore 45 may also define a countersink disposed at a distal end of the elongated shaft 40. The countersink may be adapted to receive a set fastener S (such as a break-off set screw or other set screw compatible with the anchor A) for securing the vertebral rod R to the anchor A. In other embodiments, such as that shown generally in FIGS. 8-10 the bore may be sized to allow a set fastener S to pass through the bore 45 from a proximal end 47 of the elongated shaft 40 through a distal end 48 of the elongated shaft 40. As described herein, the set fastener S may comprise a break-off set screw or other set screw compatible with a threaded inner surface of the anchor A that is adapted to secure the vertebral rod R to the anchor A after the vertebral rod R has been reduced into the anchor A by the instrument 14. In such embodiments, after the final reduction of the vertebral rod R into the anchor A (see, for example, FIG. 9), the set fastener S may be passed completely through a bore 45 defined in the elongated shaft 40. In some such embodiments, the set fastener S may be pre-loaded onto the distal end of a set fastener driver 70 that may be passed completely through the bore 45 defined in the elongated shaft 40 after reduction of the rod R into the anchor A.

As described herein, the instrument 10 may also comprise a reducing element 30 (such as the reducing sleeve 30, shown generally in FIG. 1) disposed at least partially outside an outer surface of the first and second jaws 21, 22 of the gripper 20. The reducing element may be configured to engage with the vertebral rod R so as to guide the vertebral rod R towards an opening and/or housing (such as the screw “tulip”) defined in a proximal portion of the anchor A. In some embodiments, the reducing element 30 may be operably engaged directly with elongated shaft 40. For example, as shown in FIGS. 12A and 12B, the reducing element 30 may be attached directly to a distal end 48 of the elongated shaft 40.

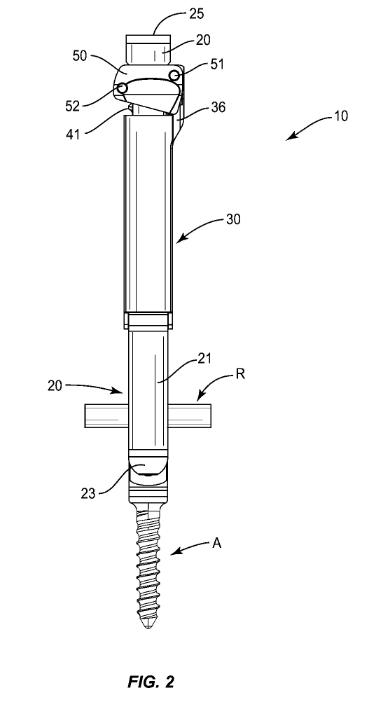

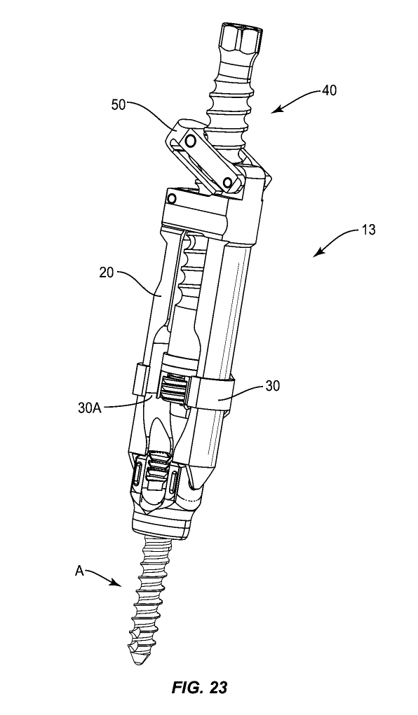

The reducing element 30 may comprise a full sleeve extending completely about the gripper 20 (and the jaws 21, 22 thereof) as shown generally in FIGS. 1-5. In some embodiments, the reducing element 30 may comprise a reducing band extending completely about the gripper 20 and having a reducing tab 32 extending distally from the reducing element 30 and configured for urging the rod R distally as the reducing element 30 is driven towards the distal ends 23, 24 of the jaws 21, 22. In other embodiments, as shown generally in FIG. 23, the reducing element 30 may extend only partially about the gripper 20. In such embodiments, the reducing element 30 may define a gap 30A co-located substantially with an anchor A capturing space formed between the jaws 21, 22 of the gripper 20. Such embodiments may provide a clinician with more working room to insert and/or preload a set fastener S (see FIGS. 2, 3 and 9) in a countersunk bore defined in a distal end 48 of the elongated shaft 40 (as described in further detail herein with respect to FIGS. 3 and 9).

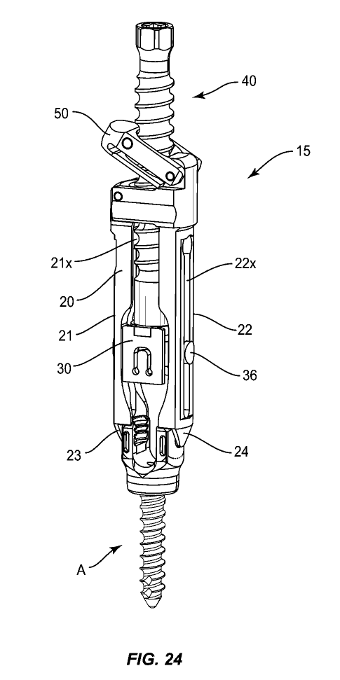

FIG. 24 shows another exemplary instrument 15 embodiment where the reducing element 30 extends only partially outside the gripper 20. In the embodiment of FIG. 24, the reducing element 30 includes tabs 36 that extend through complementary slots 21x, 22x defined in the jaws 21, 22 of the gripper. The tabs 36 have enlarged outer portions that extend outward from the tab stem so as to be disposed substantially on an outer surface of the jaws 21, 22 of the gripper. Thus, the tabs 36 may counteract a preset bias in the jaws 21, 22 (the preset being jaws 21, 22 biased away from each other) as the reducing element 30 is moved distally along the gripper 20 by urging the jaws 21, 22 towards one another to grip the anchor A therebetween as shown in FIG. 24.

The instrument 10 may also comprise a release device 50. In some embodiments, such as those shown in FIGS. 1-5, the reducing element 30, may be selectively operably engaged with the elongated shaft 40 via the release device 50. For example, as shown in FIG. 2, the instrument 10 may comprise a release device 50 disposed about the elongated shaft 40. The release device 50 may be movable between a first position and a second position relative to the elongated shaft 40.

Depending on the configuration of the instrument, the release device 50 may be operably engaged with one or more of the various components of the instrument in order to selectively engage the threads 41 of the elongated shaft 40. In some embodiments, the release device 50 may be pivotable about the gripper 20 (see, for example, FIGS. 6-8, where the elongated shaft 40 is movable relative to the gripper 20 along the longitudinal axis L). In other embodiments, the release device 50 is pivotable about the reducing element 30 (see, for example, FIGS. 1-5, wherein the elongated shaft 40 is rotatable relative to the gripper 20 but fixed relative to the gripper 20 along the longitudinal axis L).

Referring generally to FIGS. 2 and 6, the release device 50 may comprise at least one pin member 52 configured to engage the threaded outer surface 41 of the elongated shaft when the release device 50 is in the second position. In some embodiments, as shown for example, in FIG. 6, the pin member 52 may extend along a pin axis substantially orthogonal to the longitudinal axis L. The release device 50 may comprise a lever element pivotable between the first position and the second position relative to the elongated shaft 40. The lever element may pivot about a pivot pin 51. In some embodiments, as shown in FIG. 6, the pivot pin 51 may extend substantially parallel to the pin axis such that the release device 50 is pivotable between the first position and the second position relative to the elongated shaft 40. The lever element may be configured to engage the pin member 52 with the threaded outer surface of the elongated shaft 40 when the release device 50 is in the second position. Conversely, the lever element of the release device 50 may be further configured to disengage the pin member 52 from the threaded outer surface 41 of the elongated shaft 40 when the release device 50 is in the first position.

As shown in FIG. 3, the release device 50 may be operably engaged with and pivotable about a proximal end of the reducing element 30. In some embodiments, the release device 50 may include a pivot pin 51 disposed between an extension portion 36 of the reducing element 30 and the release device 50 may be embodied as a pivotable lever including a pin member 52. In such embodiments, the release device 50 lever element is configured to engage the at least one pin member 52 with the threaded outer surface 41 of the elongated shaft 40 when the release device 50 is pivoted about the pivot pin 51 and into position between adjacent threads 41 formed on the elongated shaft 40. Furthermore, as shown in the cross-section of FIG. 3, the release device 50 lever element may be further configured to disengage the at least one pin member 52 from the threaded outer surface 41 of the elongated shaft 40 when the release device 50 is pivoted proximally about the pin member 51.

Therefore, generally when the release device 50 is in a first position (see, for example, FIG. 3) relative to the threaded outer surface 41 of the elongated shaft 40, the release device 50 (and/or a pin member 52 thereof) is substantially disengaged with the threaded outer surface 41 of the elongated shaft 40 such that the reducing element 30 is slidable along the longitudinal axis L relative to the gripper 20.

In contrast, when the release device 50 is in a second position (such as, for example, pivoted counterclockwise about the pin member 51 such that the pin member 52 is seated between adjacent threads 41 formed on the elongated shaft 40), the release device 50 (and/or a pin member 52 thereof) may be threadably engaged with the threaded outer surface 41 of the elongated shaft 40 such that a rotation of the elongated shaft 40 moves the reducing element 30 along the longitudinal axis to move the vertebral rod R towards the distal ends 23, 24 of the gripper 20 and into the anchor A. According to various embodiments shown herein (see FIGS. 3 and 6, for example) the elongated shaft 40 may comprise a drive interface 44 (such as a hexagonal aperture) adapted to receive a tool (including but not limited to a hex driver) configured for rotating the elongated shaft 40 about the axis L in order to drive the reducing element 30 in a distal direction when the release device 50 is engaged with the threaded outer surface 41 of the elongated shaft 40.

Therefore, using the various embodiments of the instruments 10, 11, 12, 13, 14, 15, 16, 17, 18 and 19 described herein, a user may selectively disengage the release device 50 from the threads 41 of the elongated shaft 40 such that the reducing element 30 may be slid quickly about the gripper 20 to a position adjacent the rod R. Once the reducing element 30 is in relatively close proximity to the rod R (and/or in contact with the rod R), the release device 50 may be re-engaged with the threads 41 of the elongated member 40 which may then be rotated about the longitudinal axis L to sequentially drive the reducing element 30 distally relative to the gripper 20 to reduce the rod R into the anchor A.

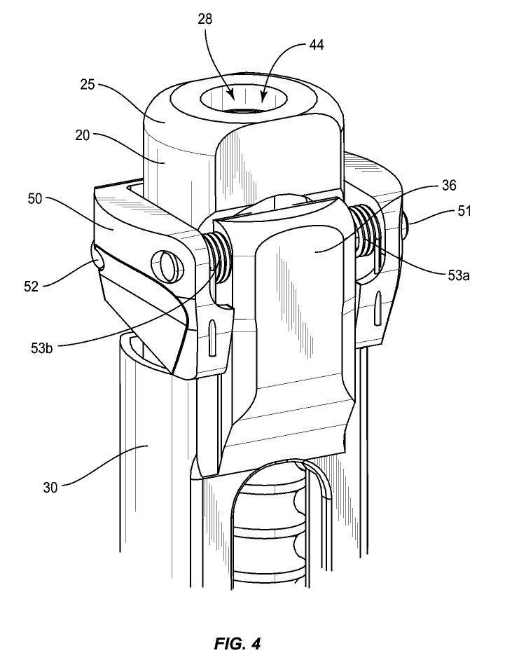

In some embodiments (see, for example, FIGS. 4 and 8), the release device 50 may comprise one or more biasing elements 53 operably engaged with the release device 50 and configured to bias the release device 50 towards at least one of the first and second positions relative to the threaded outer surface 41 of the elongated shaft 40. For example, as shown in FIG. 4, the release device 50 may comprise a pair of torsion springs 53a, 53b engaged between the pivot pin 51 and the release device 50 to bias the release device 50 (and the pin 52 carried therein) into engagement with the threads 41 formed in the elongate member 40. Thus, a user of the instrument 10 must push against the biasing action of the springs 53a, 53b to disengage the pin 52 of the release device 50 from the threads 41 of the elongated shaft 40 in order to slide the reducing element 30 into position adjacent the rod R. FIG. 6 shows an alternate embodiment of the instrument 14 wherein the pivot pin 51 is engaged with the gripper 20 such that the release device 50 is pivotable about the gripper 20. The instrument 14 also includes a biasing element 53 in the form of a single torsion spring 53 configured for biasing a pin member 52 (carried by the release device 50) into contact with the threads 41 formed on the outer surface of the elongated shaft 40 that extends within the gripper 20. In such embodiments, as shown in FIG. 11B, the gripper 20 defines a release device aperture 29 through which the release device 50 (and the pin member 52 carried thereby) may engage the threads 41 formed on the elongated shaft 40.

The release device 50 may be embodied as a lever element as described herein and shown generally in FIGS. 1-15C and FIGS. 23-27. However, the release device may also comprise a variety of other mechanisms configured for selectively engaging and disengaging a pin member 52 or other release device 50 with a threaded outer surface 41 of the elongated shaft 40.

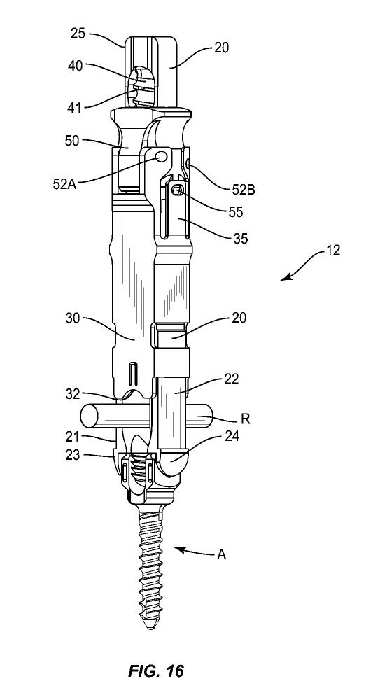

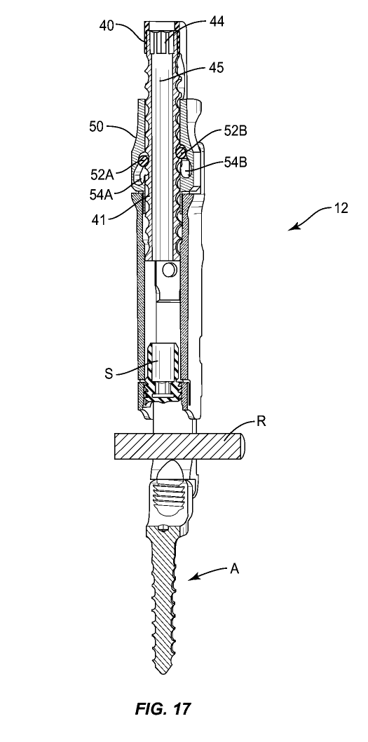

For example, the instrument 12 depicted generally in FIGS. 16-17 shows a release device 50 embodiment comprising a pair of pin members 52A, 52B that may be configured for selectively engaging the threads 41 of the elongated shaft. As shown in FIG. 16, the pin members 52A, 52B may be carried by a complementary pair of tabs extending proximally from the reducing element 30. The tabs may be resilient and/or flexible to accommodate the movement of the pin members 52A, 52B towards and/or away from the threads 41 defined on an outer surface of the elongated shaft 40. Furthermore, the pin members 52A, 52B may be operably engaged directly with the release device 50 which, as shown in the cross-section of FIG. 17, may define a pair of cam chambers 54A, 54B. As shown generally in FIG. 17, the pin members 52A, 52B may be biased away from the threads 41 (by a resilient property of the materials used in the reducing element 30 (and the tabs extending proximally therefrom). As a user of the instrument 12 pushes the release device 50 in a distal direction, the cam chambers 54A, 54B may act to urge the pin members 52A, 52B into contact with the threads 41 of the elongated shaft 40 such that rotation of the elongated shaft 40 (using the drive interface 44 shown in FIG. 17) may urge the reducing element 30 distally towards the vertebral rod R so as to reduce the rod R into the anchor A. In some embodiments, the reducing element 30 may further comprise a first locking tab 35 as shown in FIG. 16, the first locking tab defining an aperture for receiving a second locking tab 55 extending from the release device 50. The second locking tab 55 may comprise an incline structure (see FIG. 16) for releasably locking the release device 50 in the second position (as shown in FIG. 17).

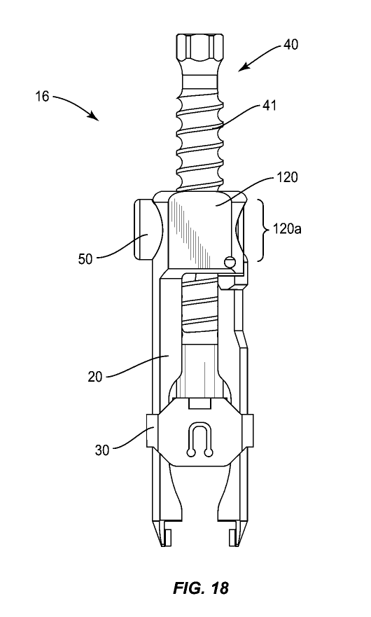

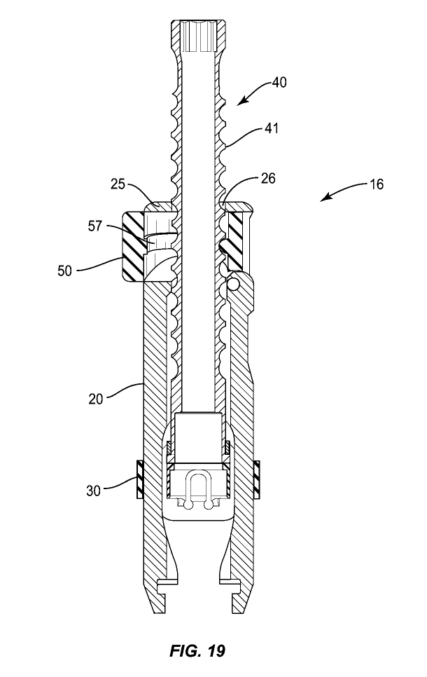

FIGS. 18-19 show an exemplary instrument 16 embodiment having a “button” style release device 50 that may be configured for selectively engaging the threads 41 of the elongated shaft. FIG. 19 shows a cross-section of the instrument 16 and the release device 50 which may define a bore through which the elongated shaft 40 may extend as shown in FIG. 19. The release device 50 may also comprise a raised portion 57 extending into the bore. As shown, for example, in FIG. 19, the raised portion 57 may comprise a semicircular cross-section or other cross-sectional shape complementary to the geometry of the threads 41 and/or suitable for seating between adjacent threads 41 defined on an outer surface of the elongated shaft 40. The release device 50 “button” may be, in various embodiments, biased towards the first and/or second positions. For example, in some embodiments, the release device 50 may comprise a biasing element (such as a coil spring, for example) disposed in the bore defined in the release device 50 for biasing the raised portion 57 either towards the threads 41 or away from the threads 41 such that a user input (i.e. the press of a button) may be required to engage and/or disengage the raised portion 57 with the threads 41. As described herein with respect to other release device embodiments, when the raised portion 57 of the release device 50 is in the first position (disengaged from the threads 41) the reducing element 30 may be freely slidable about the gripper. Conversely, when the raised portion 57 of the release device 50 is in the second position (engaged with the threads 41) the reducing element 30 may be sequentially driven by the rotation of the elongated shaft 40 relative to the gripper 20.

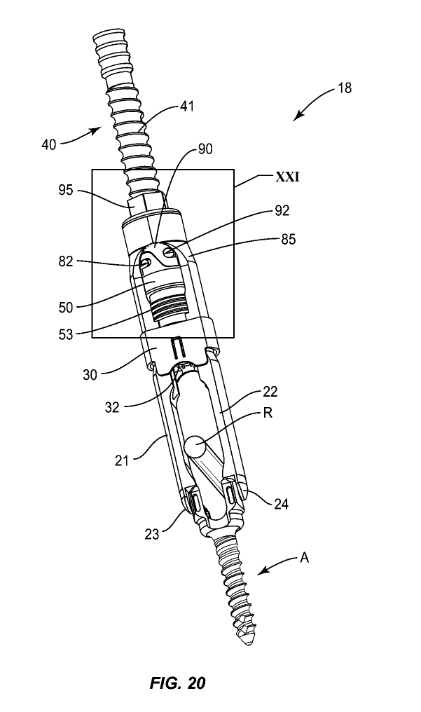

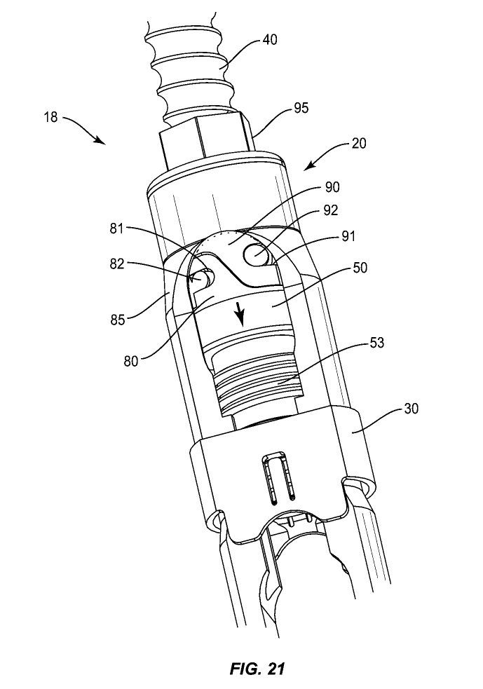

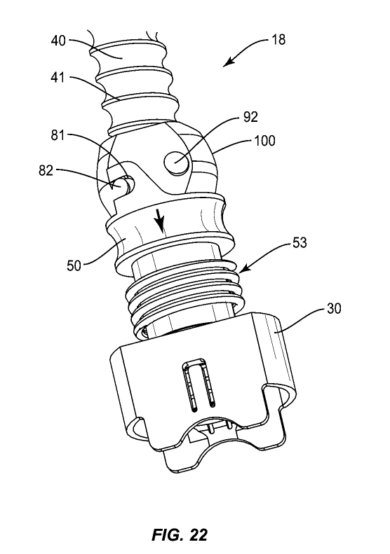

FIGS. 20-22 show an exemplary instrument 18 embodiment having a “ball” style release device wherein the ball 100, shown in FIG. 22 may comprise a pivot tab 92 (and/or an opposing pair of pivot tabs 92) and a contact member 82 configured for selectively engaging the threads 41 of the elongated shaft 40. Instrument 20 is shown fully assembled in FIG. 20 wherein the ball 100 is obscured from view by the release device 80 and sleeve 90 that may be used to orient the ball 100 relative to the elongated shaft 40 such that depression and/or release of the release device 80 may selectively engage and/or disengage the contact member 82 with the threads 41 of the elongated shaft 40. The ball 100 may be housed in a conical housing 85 disposed near a proximal end of the gripper 20 as shown generally in FIG. 21. The instrument 18 may also comprise a biasing element (such as a coil spring 53) disposed about the elongated shaft 40 for biasing the release device 50 towards one of either the first or second positions.

Referring to FIG. 21, the sleeve 90 of the instrument 18 may be fixed in place relative to the gripper 20 by a bolt 95 or other fastener such that a slot 91 defined in the sleeve 90 may restrict the freedom of motion of the pivot tab 92 extending from the ball 100. Furthermore, the release device 80 may define a cam slot 81 configured for pivoting the ball 100 about the pivot tab 92 (relative to the elongated shaft 40) such that the contact member 82 of the ball 100 may be selectively engaged and/or disengaged from the threads 41 defined on an outer surface of the elongated shaft 40. For example, in some embodiments, as shown in FIGS. 21-22, the release device 80 may be biased in a proximal direction such that the ball 100 is pushed upward and the contact member 82 is urged out of contact with the threads 41. Therefore, a user input may be required (i.e. pushing the release device 50 distally) in order to engage the contact member 82 with the threads such that the reducing element 30 is drivable by the rotation of the elongated shaft 40 relative to the gripper 20. It should be noted that the biasing member 53 may be oriented and/or constructed to reverse the bias of the release member 80 such that a user input may be required to disengage the contact member 82 from the threads 41 of the elongated shaft 40.

FIGS. 20-22 also depict an exemplary embodiment wherein the elongated shaft 40 is non-rotatable relative to the gripper 20. For example, elongated shaft 40 may be rigidly assembled with the reducing element 30, such that driving hex bolt 95 will rotate the sleeve 90 and the ball 100 about the longitudinal axis L such that the entire release device assembly is moved proximally down the elongated shaft 40 along with the reducing element 30.

As shown in FIGS. 11A-13, the various components of the instrument 14 may be selectively assembled and disassembled for cleaning, sterilization, and/or specialized procedures.

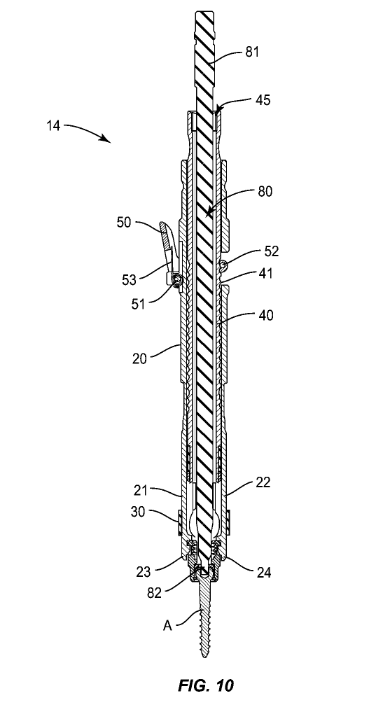

The various embodiments of the instruments 11, 12, 13, 14, 15, 16, 17, 18 and 19 described herein may also comprise accessory components suitable for passage through a bore 45 defined in the elongated shaft 40. For example, in some embodiments, such as that shown in FIG. 10, the instrument 14 may further comprise a driver 80 configured to be insertable through the bore 45. As shown in FIG. 10, the driver 80 may be configured to cooperate with the gripper 20 to attach the anchor A to a vertebral member. For example, in some embodiments, the anchor A may be “preloaded” between the jaws 21, 22 of the gripper 20 (which may comprise toothed distal ends 23, 24 configured to cooperate with apertures defined in a proximal end of the anchor A to grip the anchor A). As shown in FIG. 10, the reducing element 30 may be advanced distally along the gripper 20 to urge the jaws 21, 22 into close contact with the anchor A. The driver 80 may be inserted through the bore 45 defined in the elongated shaft such that a working tip 82 of the driver 80 is inserted into the anchor A. The complete subassembly of the driver 80, instrument 14, and anchor A may then be placed adjacent a vertebral member such that the instrument 14 and the driver 80 may be used in combination to drive the anchor A directly into the vertebral body. The driver 80 may comprise a proximal driver attachment 81 suitable for engagement with powered surgical tools and/or handle attachments (such as a t-grip driver handle). The working tip 82 of the driver 80 may also comprise a variety of male and female driver elements, including but not limited to: hex drivers, torx drivers, ratchets, screwdrivers and combinations thereof.

In some embodiments, as shown in FIG. 9, the instrument 14, may also comprise a set fastener driver 70 configured to be insertable through the bore 45 defined in the elongated shaft 40. As described herein, the bore may be sized in some embodiments to pass a set fastener S completely through the bore 45 from a proximal end 47 of the elongated shaft 40 through the bore and out a distal end 48 of the elongated shaft 40. In other embodiments, as shown in FIG. 3, the set fastener S may be preloaded into a distally-located countersink within the elongated shaft 40. In any of these embodiments, a set fastener driver 70 may be inserted through the bore 45, wherein the set fastener driver 70 may be configured to drive a set fastener S into a head (such as a “tulip” of a pedicle screw) of the anchor A to secure the vertebral rod R within the anchor A. As shown in FIG. 9, the set fastener driver 70 may comprise a break-off driver suitable for applying substantial torque to a head of a set fastener S to cleanly break off a head of the set fastener S after it is seated in the anchor A. The set fastener driver 70 may also comprise a variety of male and female driver elements, including but not limited to: hex drivers, torx drivers, ratchets, screwdrivers and combinations thereof.

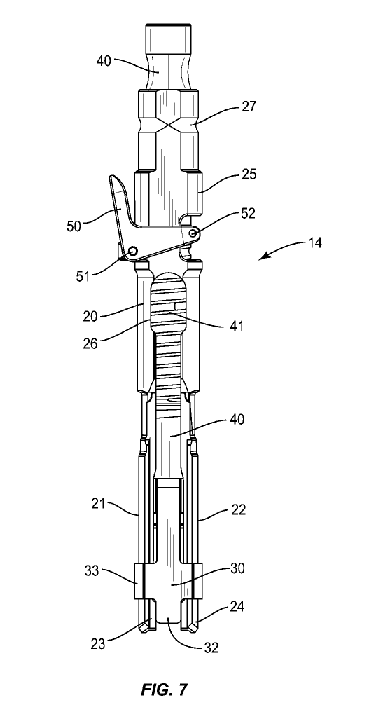

As shown in FIGS. 6 and 7, some embodiments of the instrument 14 may further comprise attachment points 27 for vertebral column manipulation (VCM) instrumentation. Exemplary VCM instruments include the CD HORIZON™ VCM Instrument Set available from Medtronic Spinal and Biologics in Memphis, Tennessee. In such embodiments, multiple instruments 14 may be attached to a corresponding number of anchors A disposed in vertebral members. A clinician may then attach VCM bridge members, brackets and handles to the attachment points 27 in order to utilize the instrument 14 as a VCM component instrument in cases where a patient is suffering from spinal deformity and/or trauma requiring VCM procedures.

Various methods for moving a vertebral rod R into a bone anchor A attached to a vertebral member (not shown) are also provided herein. Referring to FIG. 1, in one embodiment, the method comprises positioning the vertebral rod R within a capturing space formed between a reducing element 30 and an opposing first and second jaws 21, 22 of a gripper 20 that are attached to the bone anchor A. As described herein, the reducing element 30 may be disposed at least partially about an outer surface of the first and second jaws 21, 22 of the gripper 20.

The method further comprises sliding the reducing element 30 along a longitudinal axis L towards the vertebral rod R. The longitudinal axis L extends between the opposing first and second jaws 21, 22. As described herein, the sliding step may be accomplished by disengaging a release device 50 disposed about a threaded outer surface 41 of an elongated shaft 40, wherein the elongated shaft 40 is disposed at least partially within the gripper 20. The method further comprises engaging the reducing element 30 with the vertebral rod R (which may comprise simply sliding the reducing element to a position substantially adjacent to the rod R).

The method further comprises reducing the vertebral rod R into the bone anchor A by engaging the release device 50 with the threaded outer surface 41 of the elongated shaft 40 and rotating the elongated shaft 40 relative to the gripper 20 to move the reducing element 30 along the longitudinal axis L towards the bone anchor A. The method may also comprise, in some embodiments, rotating the release device 50 (or some portion thereof) relative to the elongated shaft 40 (which may be rotatably fixed relative to the gripper 20) in order to move the reducing element 30 along the longitudinal axis L towards the bone anchor A. Thus, when the release member 50 is in the second position, the method may more generally comprise moving the release device 50 and the elongated shaft 40 relative to one another so as to reduce the rod R into the anchor A via the motion of reducing element 30. In some embodiments, the method further comprises passing a set fastener through a bore defined in the elongated shaft and securing the set fastener to the bone anchor in order to secure the vertebral rod R to the anchor A.

In one embodiment, the method embodiments may be performed percutaneously. In other embodiments, the method may be performed with an open approach, semi-open approach, or a muscle-splitting approach.

Spatially relative terms such as “under”, “below”, “lower”, “over”, “upper”, and the like, are used for ease of description to explain the positioning of one element relative to a second element. These terms are intended to encompass different orientations of the device in addition to different orientations than those depicted in the figures. Further, terms such as “first”, “second”, and the like, are also used to describe various elements, regions, sections, etc and are also not intended to be limiting. Like terms refer to like elements throughout the description.

As used herein, the terms “having”, “containing”, “including”, “comprising” and the like are open ended terms that indicate the presence of stated elements or features, but do not preclude additional elements or features. The articles “a”, “an” and “the” are intended to include the plural as well as the singular, unless the context clearly indicates otherwise.

Also, it is understood that each of the above-described embodiments may be combined in whole or in part with one or more of the other above-described embodiments. It is further understood that each of the above-described embodiments may be combined in whole or in part with other components, devices, systems, methods and/or surgical techniques known to those skilled in the art to provide spinal stabilization and/or vertebral column manipulation (VCM).

The embodiments disclosed herein may be carried out in other specific ways than those herein set forth without departing from the scope and essential characteristics of the disclosure. The present embodiments are, therefore, to be considered in all respects as illustrative and not restrictive, and all changes coming within the meaning and equivalency range of the appended claims are intended to be embraced therein.

What is claimed is:

1. An instrument for moving a vertebral rod into an anchor attached to a vertebral member, the instrument comprising:
a gripper including opposing first and second jaws disposed about a longitudinal axis, each of the opposing first and second jaws having a distal end to attach to the anchor, and a proximal end;
an elongated shaft extending along the longitudinal axis and operably engaged with the gripper, the elongated shaft including a threaded outer surface;
a reducing element disposed about the first and second jaws of the gripper and configured to engage with the vertebral rod; and
a release device disposed about the elongated shaft, the release device being movable between a first position and a second position relative to the elongated shaft;
wherein when the release device is in the first position, the release device is substantially disengaged from the threaded outer surface of the elongated shaft such that the reducing element is slidable along the longitudinal axis relative to the gripper; and
wherein when the release device is in the second position, the release device is threadably engaged with the threaded outer surface of the elongated shaft such that a relative movement of the elongated shaft and the reducing element along the longitudinal axis moves the vertebral rod towards the distal ends of the gripper and into the anchor.

2. The instrument of claim 1, wherein the opposing first and second jaws of the gripper are biased away from one another, and wherein the reducing element is configured to urge the distal ends of the opposing first and second jaws towards one another to grip the anchor therebetween as the reducing element is moved towards the distal ends of the gripper.

3. The instrument of claim 1, further comprising a biasing element operably engaged with the release device and configured to bias the release device towards the second position.

4. The instrument of claim 1, further comprising a biasing element operably engaged with the release device and configured to bias the release device towards the first position.

5. The instrument of claim 1, wherein the elongated shaft defines a bore extending therethrough along the longitudinal axis.

6. The instrument of claim 5, wherein the bore defines a countersink disposed at a distal end of the elongated shaft, the countersink adapted to receive a set fastener for securing the vertebral rod to the anchor.

7. The instrument of claim 5, wherein the bore is sized to allow a set fastener to pass through the bore from a proximal end of the elongated shaft through a distal end of the elongated shaft, the set fastener adapted to secure the vertebral rod to the anchor.

8. The instrument of claim 1, wherein the elongated shaft is movable relative to the gripper along the longitudinal axis.

9. The instrument of claim 8, wherein the gripper comprises a transverse member at the proximal end, the transverse member connecting the first and second jaws, the transverse member defining an aperture through which the elongated shaft at least partially extends.

10. The instrument of claim 8, wherein the release device is pivotable about the gripper.

11. The instrument of claim 1, wherein the elongated shaft is substantially fixed relative to the gripper along the longitudinal axis, and wherein the elongated shaft is rotatable relative to the gripper about the longitudinal axis.

12. The instrument of claim 10, wherein the release device is pivotable about the reducing element.

13. The instrument of claim 1, wherein the release device comprises at least one pin member configured to engage the threaded outer surface of the elongated shaft when the release device is in the second position.

14. The instrument of claim 13, wherein the release device comprises a lever element pivotable between the first position and the second position relative to the elongated shaft, the lever element configured to engage the at least one pin member with the threaded outer surface of the elongated shaft when the release device is in the second position, the lever element further configured to disengage the at least one pin member from the threaded outer surface of the elongated shaft when the release device is in the first position.

15. The instrument of claim 5, further comprising a driver configured to be insertable through the bore, the driver configured to cooperate with the gripper to attach the anchor to the vertebral member.

16. The instrument of claim 5, further comprising a set fastener driver configured to be insertable through the bore, the set fastener driver configured to drive a set fastener into a head of the anchor to secure the vertebral rod to the anchor.

17. The instrument of claim 1, wherein the gripper comprises at least one attachment point adapted to operably engage at least a portion of a vertebral column manipulation device.

18. A method of moving a vertebral rod into a bone anchor attached to a vertebral member, the method comprising:
positioning the vertebral rod within a capturing space formed between a reducing element and an opposing first and second jaws of a gripper that are attached to the bone anchor, the reducing element disposed at least partially about an outer surface of the first and second jaws of the gripper;
sliding the reducing element along a longitudinal axis towards the vertebral rod, the longitudinal axis extending between the opposing first and second jaws, by disengaging a release device disposed about a threaded outer surface of an elongated shaft, the elongated shaft being operably engaged with the gripper;
engaging the reducing element with the vertebral rod; and
reducing the vertebral rod into the bone anchor by engaging the release device with the threaded outer surface of the elongated shaft and moving the release device and the elongated shaft relative to one another to move the reducing element along the longitudinal axis towards the bone anchor.

19. The method of claim 18, further comprising:
placing the anchor into position between the first and second jaws of the gripper;
sliding the reducing element along the longitudinal axis towards the anchor such that the reducing element urges the first and second jaws towards one another to grip the anchor therebetween;
inserting a driver through a bore defined in the elongated shaft, the bore extending along the longitudinal axis, such that the driver operably engages a proximal end of the anchor; and
rotating the gripper and the driver such that the gripper and driver cooperate to drive the anchor into the vertebral member.

20. An instrument for moving a vertebral rod into an anchor attached to a vertebral member, the instrument comprising:
a gripper including opposing first and second jaws disposed about a longitudinal axis, each of the opposing first and second jaws having a distal end to attach to the anchor, and a proximal end;
an elongated shaft extending along the longitudinal axis and operably engaged with the gripper, the elongated shaft including a threaded outer surface;
a reducing element disposed about the first and second jaws of the gripper and configured to engage with the vertebral rod; and
a release device disposed about the elongated shaft, the release device including at least one pin member extending along a pin axis substantially orthogonal to the longitudinal axis, the release device being pivotable between a first position and a second position relative to the elongated shaft;
wherein when the release device is in the first position, the pin member is substantially disengaged from the threaded outer surface of the elongated shaft such that the reducing element is slidable along the longitudinal axis relative to the gripper; and
wherein when the release device is in the second position, the pin member is engaged between a pair of adjacent threads disposed on the threaded outer surface of the elongated shaft such that a relative movement of the elongated shaft and the reducing element along the longitudinal axis moves the vertebral rod towards the distal ends of the gripper and into the anchor.
